(19) United States
(12) Patent Application Publication
(10) Pub. No.: US 2025/0025662 A1
(43) Pub. Date: Jan. 23, 2025

(54) INTERACTIVE EMOTIONAL SUPPORT DEVICE AND METHODS

(71) Applicant: Linda J. CRONIN, Brewster, MA (US)

(72) Inventor: Linda J. CRONIN, Brewster, MA (US)

(21) Appl. No.: 18/714,595

(22) PCT Filed: Dec. 1, 2022
(86) PCT No.: PCT/US2022/051473

§ 371 Date: May 30, 2024

Related U.S. Application Data
(60) Provisional application No. 63/264,716, filed on Dec. 1, 2021.

(51) Int. Cl. A61M 21/02; A61M 21/00; G16H 20/00

(52) U.S. Cl. CPC A61M 21/02; G16H 20/00; A61M 2021/0016; A61M 2021/0022; A61M 2021/0027; A61M 2021/0044; A61M 2021/0066; A61M 2205/3553; A61M 2205/3584

(57) ABSTRACT

A stuffed, interactive children's toy for assisting individuals in self-calming and/or self-regulating exercises. The toy may include one or more weights, a microphone, a motor, one or more light emitting diodes (LEDs), and/or one or more speakers. The toy optionally further includes one or more buttons corresponding with one or more emotional states. For example, a red button, a yellow button, and/or a green button. Upon pressing the button, the toy may deliver either a random and/or a preprogrammed intervention. The toy may be used in combination with a children's book. In another example, the toy may be in communication with a mobile application (app) via a Bluetooth connection. In yet another example, the toy may be in communication with a website and/or a remote-control device. The app may be used to record emotional data, physical date, and/or to assign preprogrammed stimuli to the one or more buttons.

### CROSS-REFERENCE TO RELATED APPLICATION

This application claims the benefit of U.S. Provisional Application No. 63/264,716 filed Dec. 1, 2021, which is hereby incorporated by reference.

### BACKGROUND OF THE INVENTION

This present disclosure generally relates to interactive devices, such as toys. More specifically, the present disclosure pertains to devices to help an individual self-regulate (e.g., self-sooth, calm down) a sympathetic nervous system (emotional) response by regaining balance in the autonomic nervous system.

Individuals, especially adults and children with special needs and/or emotional troubles, may go through an emotional episode. The episode may include multiple stages of emotion. For example, a child may escalate from a calm state to a fight or flight state suddenly and/or without warning. Once a child has reached the fight or flight state, the child may have difficulty self-soothing and/or self-calming back down to a calm state. Current stress relief toys such as stress balls, fidget toys, and/or meditation toys provide some relief; however, reducing or limiting the amount of time a child is in a fight or flight and/or upset state is desirable. Thus, there is a need for improvement in this field.

### SUMMARY OF THE INVENTION

In certain aspects, the present disclosure pertains to interactive toys. More preferably, the interactive toy is a therapeutic healing toy useful to teach an individual (e.g., a child) how to self-regulate (e.g., self-soothe, calm down), by regaining balance in the autonomic nervous system, through a system of interventions and rewards. The toy is configured to provide interventions aimed at stimulating the vagus nerve, which is one of the major nerves feeding this system, and continues with interventions designed to promote the switch from the sympathetic nervous system (fight or flight) to the parasympathetic nervous system (rest and digest) and so back to equilibrium, with rewards given for reaching this preferred state. With the autonomic nervous system in balance, the neocortex (thinking portion of the brain) comes back on line and executive functioning capabilities are accessible and the individual is now back in a state of calm. In this state the individual is more open and capable of digesting the lessons that can be learned in this moment. This system of interventions and rewards will aid the individual to eventually learn to self-regulate and/or lower the incidences of dysregulation in the first place. In this state of autonomic nervous system balance the individual is more able to learn to regulate emotions as they come, to learn in school, to better practice mindfulness, meditation, etc. to reduce suffering and greatly increase their quality of life.

In one aspect, the interactive toy is a stuffed toy. The interactive toy may be in any of a variety of shapes and sizes. For example, the interactive toy may be a brain-shaped toy. Preferably the toy weighs at least 3 pounds (lbs.) and/or at least 5 pounds (lbs.). The toy may include one or more speakers, headphone jacks, vibrating motors, lights, and/or microphones located within the toy. In another example, the toy may include a fabric patch for adding aromatherapy essential oils to the toy. The fabric patch may be removable for cleaning. For example, it may be attached to the toy with hook and loop fasteners. In another example, the toy may include a warming pad for generating heat to the toy. In another example, the toy may include a removable and washable cover for protecting the toy from dust.

The toy may include one or more interactive inputs that, as will be explained below, may correspond to different emotional states and provide an associated intervention. For example, the toy may include a first zone that corresponds to a “fight or flight” emotional state. The toy may include interactive buttons having different colors, shapes, sizes, and/or textures and/or that are located in separate areas of the toy. In particular arrangements, the toy may include red, yellow, and/or green zones.

The inputs initiate different interventions that may be visual, audible, and/or tactile in nature. For example, pressing a red button may initiate a calming intervention. A calming intervention may include: vibration, humming sounds, rhythmic beating sounds (e.g., a heartbeat sound), rainfall sounds, ocean wave sounds, flowing stream sounds, babbling brook sounds, binaural beats meditation sounds, guided exercises such as focus exercises (e.g., “name three things you see”, “name two things you hear”, “name one thing you smell”), guided breathing exercises (e.g., “empty your lungs of air, breath in through your nose and fill your belly for 4 . . . 3 . . . 2 . . . 1 . . . , hold your breath for 3 . . . 2 . . . 1 . . . , now blow out of your mouth for 7 . . . 6 . . . 5 . . . 4 . . . 3 . . . 2 . . . 1 . . . ”), guided physical exercises (e.g., crossing the midline exercise such as butterfly taps, cross marches, and/or windmills; figures eights; and/or jumping jacks), guided activities with the toy (e.g., hugging and/or petting the toy), guided muscle relaxation (e.g., progressive muscle relaxation from head-to-toe or vice versa), guided counting exercise (e.g., “one hangry hippopotamus, two hangry hippopotamuses, three . . . ”), and/or other calming strategies. As a further example, once out of a fight or flight state, pressing a yellow button may initiate an intervention to aid a user in processing the situation. An intervention to aid a user in processing the situation may include: guiding the user to tell a story of what happened to make them feel the way they feel (e.g., “name it to tame it”), guided visualization (e.g., guiding the user to imagine they are in their safe place and, optionally, describe it using all senses), guiding the user through an acupressure technique such as Emotional Freedom Technique (EFT), guiding a user to repeat positive statements (e.g., “I'm loved, I'm safe, I'm wanted, I'm worthy, and everything's going to be ok”), guiding the user through breathing exercise, guiding the user to journal, telling the user a joke, and/or other processing strategies. The intervention may be arranged to reward the user once back in a calm state. For example, pressing a green button may initiate an intervention to reward the user. An intervention to reward the user may include: a light show with or without congratulations, playing a song for the user (e.g., the user's favorite song or a new song), telling a story to the user, telling a joke to the user, and/or other rewards. The toy may deliver one or more random and/or preprogrammed interventions in response to one or more inputs, such as an interactive input, being selected.

A preprogrammed intervention may be programmed via a mobile application (app) on a mobile device. In other examples, an intervention may be preprogrammed via a website and/or a remote control. Generally, the toy may be connected to one or more mobile devices via a wireless connection (e.g., Bluetooth, IEEE 802.11, near-field communication, and/or infrared communication) to facilitate programming. The toy may also share diagnostic and behavioral statistics/information with the app via the wireless connection. For example, the app may record the number of times and/or amount of time that the device provided intervention after and/or between inputs (e.g., the time a user was in the red, yellow, and/or green zones and/or the amount of time it took for a user to transfer from one zone to another such as from red to yellow). In other examples, the toy may include a heartrate monitor.

In another aspect, the user may create (e.g., record) personal interventions and assign the personal interventions to one or more states (e.g., buttons) on the toy using the app. For example, a parent may record themselves telling a story, singing, and/or guiding one through a meditation or an EFT tapping, just to name a few non-limiting examples. The device may be programed to play the recorded intervention (e.g., when a child presses the yellow button).

In another aspect, the toy includes one or more fasteners. The fasteners work to secure the toy in a unitary arrangement for play. The fasteners may also be arranged to fasten the toy around the neck of a child in a travel pillow arrangement. As should be appreciated, all the disclosed features may be available in the toy and/or travel pillow arrangements.

In an example use case, a child begins to feel upset and moves into the yellow zone. The yellow zone generally corresponds with the limbic system, between the fight or flight response and a calm state. When the child begins to feel stressed in the yellow zone, the child presses the yellow button and/or yellow zone of the toy. The toy then distributes either a random intervention saved to a memory of the toy and/or a preprogrammed intervention assigned to the yellow button via the app. As should be appreciated, the intervention may be any form of physical response and/or audio response. For example, the intervention may be vibration, a song, a calming voice, a game, a question, instructions, positive affirmations, and/or any other intervention integrated into the toy via the app.

In another aspect, the toy accompanies a written book written to assist individuals with self-calming strategies. In one aspect, the book may include real life situations and applications of self-soothing and/or calming strategies and how to use the strategies to self-sooth and/or calm.

The body of the toy is generally made from a soft, plush material with an interior of the toy being stuffed to promote comfort. The toy may include a body with one or more eyes, one or more legs, and/or one or more arms. The body of the toy may be generally brain-shaped in either a generally anatomically correct shape and/or in a general brain shape to increase appeal. The toy may be shaped in the form of a character from the accompanying book.

In some examples, different areas of the toy may be associated with emotional states via coloring. For example, the eyes may be colored green to represent calm and/or happy emotions (parasympathetic responses), the legs may be colored yellow to represent limbic system emotions, and the arms may be colored red to represent fight or flight emotions (sympathetic responses). As should be appreciated, the coloring of the eyes, legs, and/or arms and/or different portions of the brain (e.g., amygdala, prefrontal cortex, etc.) may be any color. In some instances, the coloring of the toy area(s) may be provided by a light transmissive window portion of the toy allowing allow light from one or more light emitters (e.g., light emitting diodes) to illuminate the area. As should be appreciated, the light emitters may emit colored light such as red, yellow, and/or green. Preferably, the light color for one or more areas may be selectable by a user.

The toy may also be depicted in a children's book. In one example, the book may be titled “Thinker's Search for Peace”, “Thinker Thinks Too Much”, and/or Jack's Magical Trip and may detail the adventures of Jack and/or Thinker (the toy) and his methods and steps for self-calming in difficult situations. In another example, the book and toy may be sold as a unitary bundle.

The toy may include one or more fasteners. The fasteners may be any form of snaps, clips, buttons, friction fit fasteners, hook and loop fasteners, zippers, and/or any combination thereof. The fasteners are generally used to secure the toy in a play arrangement. However, the fasteners may also be used to move the toy into a travel pillow arrangement. In a travel pillow arrangement, the user may undo the fasteners and place the toy around the neck. As should be appreciated, all of the arrangements disclosed herein can be available in the travel pillow and/or the play arrangements.

The toy may include a controller. The controller may be a microcontroller used to operate and control the toy. The controller may also control one or more optional accessories of the toy. The accessories may include lights, speakers, an electric motor, a microphone, and/or a wireless connection.

The toy may optionally include one or more weights. The weights are generally inserted into the toy to a total weight of at least 3 pounds (lbs.). In other examples, the total weight may be at least 5 pounds (lbs.). In one example, the weights are removable from the toy to adjust the total weight based on user preference. As was described previously, the toy may include one or more lights. The lights may be used to illuminate sections of the toy. The toy may further include one or more speakers. The speakers are optionally inserted into the toy to play music, recordings, and/or other audio. The toy may optionally include a motor. The motor is generally used to generate vibration and/or haptic feedback within the toy. For example, the toy may include a switch to enable/disable vibration as needed. The toy may optionally include a microphone. The microphone enables a user to command the toy and/or enables the toy to detect audio feedback from a user. The toy may include a wireless connection. The wireless connection may be in the form of a Bluetooth, IEEE 802.11, near-field, and/or infrared communication connection. The wireless connection enables a user and/or parent of a user to connect to the toy via a mobile device. For example, the wireless connection may enable a user and/or parent of a user to connect to the speakers via a mobile device. A user may connect the toy to a mobile application (app) to adjust settings and/or program stimuli.

The toy may optionally include one or more regions and/or zones corresponding to different emotional states. The first region generally corresponds to a fight or flight emotion, the second region generally corresponds to a limbic system emotion, and the third region generally corresponds to a calm/happy emotion. As has been mentioned previously, the regions may be color coded. For example, the first region may be red, the second region may be yellow, and the third region may be green.

The toy may optionally include one or more buttons. Generally, the toy includes three buttons corresponding to three emotional states. The first button generally corresponds to a fight or flight emotion, the second button generally corresponds to a limbic system emotion, and the third button generally corresponds to a calm/happy emotion. As has been mentioned previously, the buttons may be color coded. For example, the first button may be red, the second button may be yellow, and the third button may be green.

As should be appreciated, the buttons may be associated with the zones. For example, the first region may include the first button, the second region may include the second button, and the third region may include the third button. As has been mentioned previously, the buttons may be color coded to match the region. For example, the first button may be red, the second button may be yellow, and the third button may be green. Generally, the buttons work to activate an intervention from the corresponding region's emotional state. For example, pressing the third button in the third region would give a reward, such as a joke, story, favorite song, and/or other reward.

An app is generally located on a mobile device. In other examples, the app may be located on a website. In yet another example, the app may be on a remote-control device. The mobile device may be a cell phone, tablet, computer, and/or any combination thereof. The app may include a button corresponding to the first region, a button corresponding to the second region, and/or a button corresponding to the third region. The buttons may be used to program and/or assign stimuli to each region. For example, clicking the button for the first region enables a user to assign intervention/stimuli to the first region. Optionally, the user may also remove stimuli from the first region and/or set the region to alternate between certain stimuli randomly.

The app may also include a button for recording personal stimuli. For example, the user may record an audio clip of themselves reading a book, singing a song, saying positive affirmations, and/or any combination thereof. The recorded clip may then be assigned to one of the regions and used as an intervention.

The app may include a trend button showing statistical information related to the amount of times a child was in each zone, the amount of time it took a child to transfer into the next zone, the heart rate of the child in each zone, and other data related to behavioral statistics. As should be appreciated, the toy may upload this data to the app instantaneously and/or in a batch upload process once per day.

The app may include a settings button that enables a user to adjust the settings of the toy. For example, the user may change the vibration intensity of the toy, the light brightness and/or color, check toy battery life, and/or adjust other settings such as the duration and/or number of interventions in a particular state and/or whether the toy transitions to another state.

In some situations, a child may be in a dysregulated and/or fight or flight emotional state. For example, the child may be angry, upset, sad, and/or feeling a similar emotion. At this stage, the child, parent, and/or caregiver can press the button in the first and/or red region. Generally, the button will be either illuminated red via one or more lights and/or a red colored material. The controller of the toy determines whether the toy is set-up to deliver a preprogrammed intervention via settings on the app. If not, the child receives a randomly generated intervention. The random intervention may be vibration, calming music, meditative music, nature sounds, instructions to narrow focus, deep breathing exercise, crossing the midline exercises, instructions to perform manual exercise, hugging/petting the toy, progressive muscle relaxation, meditation, counting exercises, and/or other calming strategies. If the toy is programmed to deliver a preprogrammed intervention, the preprogrammed intervention is delivered.

Eventually the child will begin to calm and transfer from the fight or flight emotional response down into a calmer response wherein the child may press the button in the second and/or yellow region. In other examples, a parent and/or caregiver may press the button via remote control. In some examples, the child may transfer from the red or fight or flight response directly to the green or calm emotional state. In other examples, the child may remain in the fight or flight state and restart the calming process.

The controller sends the emotional transition information to the app. The controller may send information related to the number of times the child was in each zone and/or the amount of time for the child to transfer out of the zone.

When a child feels a transition in mood from a green state to a yellow state the child presses the button in the second or yellow region. Generally, the button will be either illuminated yellow via one or more lights and/or a yellow colored material. The controller of the toy determines whether the toy is set-up to deliver a preprogrammed intervention via settings on the app. If not, the child receives a randomly generated intervention. The random intervention may be telling a story, imagining a safe place, EFT tapping, positive affirmations, deep breathing exercises, motivational statements, journaling prompts, jokes, and/or other calming strategies. If the toy is programmed to deliver a preprogrammed intervention, the preprogrammed intervention is delivered.

The child begins to calm and transfer from the limbic emotional response down into the green or calm/happy system response. In some examples, the child may remain in the limbic state and restart the calming process. In other examples, the child may be unable to calm themselves and escalate into the red or fight or flight response.

The controller sends the emotional transition information to the app. The controller may send information related to the number of times the child was in each zone and/or the amount of time for the child to transfer out of the zone.

When the child is in the green or calm emotional state the child presses the button in the third or green region. In other examples, a parent and/or caregiver may press the button via remote control. Generally, the button will be either illuminated green via one or more lights and/or a green colored material. The controller of the toy determines whether the toy is set-up to deliver a preprogrammed reward via settings on the app. If not, the child receives a randomly generated reward. The random reward may be a light show with congratulations, a favorite song, a new song, a story, jokes, and/or other rewards. If the toy is programmed to deliver a preprogrammed reward, the preprogrammed reward is delivered.

The controller sends the emotional transition information to the app. The controller may send information related to the number of times the child was in each zone and/or the amount of time for the child to transfer out of the zone.

When a user wants to record a personal intervention, the user selects the record function within the app. The user records a personal intervention and/or reward. For example, the user may record a song, reading a book, praise, and/or any other intervention and/or reward. The app saves a recording of the intervention/reward. The user selects a region to assign the intervention/reward to. For example, the user may assign the intervention to the first and/or red region to help in self-calming. The user confirms the assignment of the intervention/reward to the region.

Any of the inventive aspects and embodiments discussed herein may be used independently or in combination with each other.

Other aspects, objectives, and advantages of the present disclosure will become more apparent from the following detailed description when taken in conjunction with the accompanying drawings. Further forms, objects, features, aspects, benefits, advantages, and embodiments of the present invention will become apparent from a detailed description and drawings provided herewith.

### BRIEF DESCRIPTION OF THE DRAWINGS

FIG. 1 shows an example of a stuffed brain-shaped toy.

FIG. 2 shows a top view of an example stuffed brain-shaped toy of FIG. 1.

FIG. 3 shows a diagrammatic view of the stuffed brain-shaped toy of FIG. 1.

FIG. 4 shows a diagrammatic view of a mobile application associated with the toy of FIG. 1.

FIG. 5 shows a flowchart of a red to yellow calming transition using the toy of FIG. 1.

FIG. 6 shows a flowchart of a yellow to green calming transition using the toy of FIG. 1.

FIG. 7 shows a flowchart of a reward mechanism using the toy of FIG. 1.

FIG. 8 shows a flowchart of an app programming process for the toy of FIG. 1.

### DETAILED DESCRIPTION OF THE INVENTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. It will be apparent to those skilled in the relevant art that some features may not be shown for the sake of clarity.

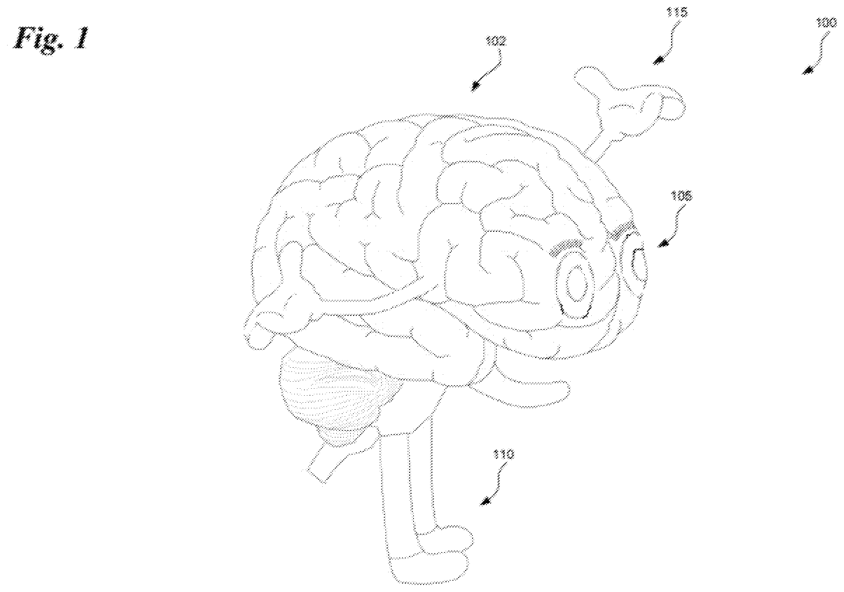

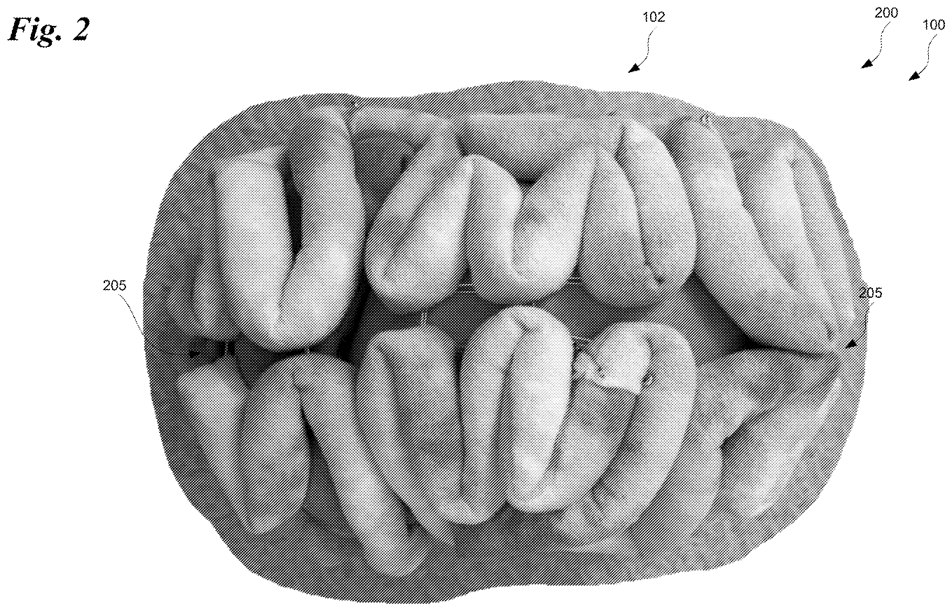

Illustrated in FIGS. 1 and 2 are examples of a brain shaped toy 100. The toy 100 generally includes a body 102 with one or more eyes 105, one or more legs 110, and one or more arms 115. The body 102 of the toy 100 is generally made from a soft, plush material with an interior of the toy 100 being stuffed to promote comfort. The body 102 of the toy is generally brain-shaped in either a generally anatomically correct shape and/or in a general brain shape to increase appeal. The body of the toy is customizable in terms of size and character.

In some examples, the eyes 105, legs 110, and arms 115 may be associated with emotional states via coloring. For example, the eyes 105 may be colored green to represent calm and/or happy emotions (parasympathetic responses), the legs 110 may be colored yellow to represent limbic system emotions, and the arms 115 may be colored red to represent fight or flight emotions (sympathetic responses). As should be appreciated, the coloring of the eyes, legs, and/or arms may be changed and/or switched based on user preference. For example, the eyes 105 may be red, the legs 110 may be green, and the arms 115 may be yellow. In other embodiments, the colors may be changed completely. For example, the eyes 105 may be purple, the legs 110 may be orange, and the arms 115 may be blue. In yet another embodiment, the eyes, legs, and/or arms may not be colored and instead include a light transmissive window portion. The window portion is used to allow light from one or more light emitting diodes (LEDs) to illuminate the eyes, legs, and/or arms. As should be appreciated, the lights may correspond to desired colors such as red, yellow, and green. In other examples, the light color may be programmable based on user preference.

The toy 100 may also be depicted in a children's book. In one example, the book may be titled “Thinker's Search for Peace” and may detail the adventures of Thinker (the toy 100) and his methods and steps for self-calming in difficult situations. In another example, the book and toy 100 may be sold as a unitary bundle. In another example, the book may be titled “Positopia World.”

As perhaps best shown in FIG. 2, the toy 100 may include one or more fasteners 205. The fasteners 205 may be any form of snaps, clips, buttons, friction fit fasteners, hook and loop fasteners, zippers, and/or any combination thereof. The fasteners 205 are generally used to secure the toy 100 in a play arrangement 200. However, the fasteners 205 may also be used to move the toy 100 into a travel pillow arrangement (not shown for ease of illustration). In a travel pillow arrangement, the user may undo the fasteners 205 and place the toy around the neck. As should be appreciated, all of the arrangements disclosed herein can be available in the travel pillow and/or the play arrangements.

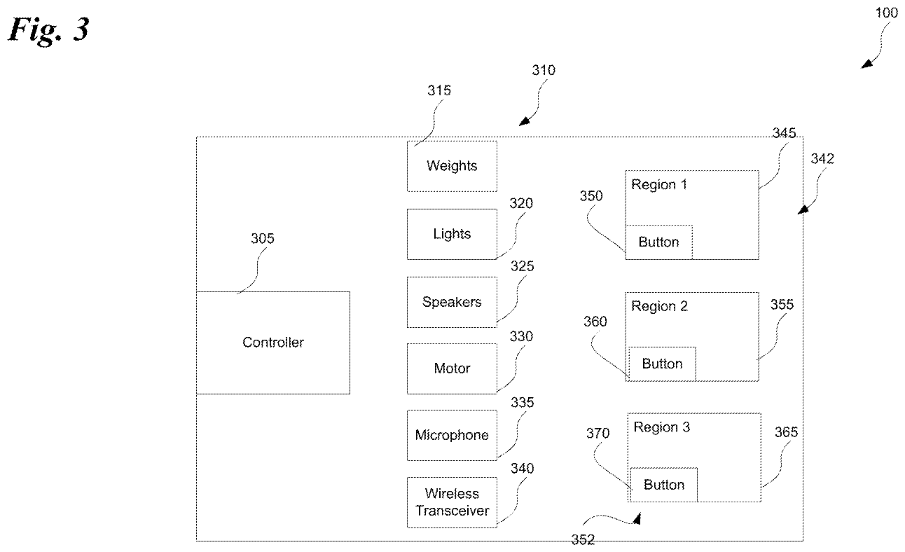

Illustrated in FIG. 3 is an example of interior components of the toy 100. The toy 100 may include a controller 305. The controller 305 may be a microcontroller used to operate and control the toy 100. The controller 305 may also control one or more optional accessories 310 of the toy 100. The accessories 310 may include lights 320, speakers 325, an electric motor 330, a headphone jack, a microphone 335, and/or a wireless connection 340.

The toy 100 may optionally include one or more weights 315. The weights 315 are generally inserted into the toy 100 to a total weight of at least 5 pounds (lbs.). In other examples, the total weight may be at least 3 pounds (lbs.). In one example, the weights 315 are removable from the toy 100 to adjust the total weight based on user preference. As was described previously, the toy 100 may include one or more lights 320. The lights 320 may be used to illuminate sections of the toy 100. The toy 100 may further include one or more speakers 325. The speakers 325 are optionally inserted into the toy 100 to play music, recordings, and/or other audio. The toy 100 may optionally include a motor 325. The motor 325 is generally used to generate vibration and/or haptic feedback within the toy 100. For example, the toy 100 may include a switch to enable/disable vibration as needed. The toy 100 may optionally include a microphone 335. The microphone 335 enables a user to command the toy 100 and/or enables the toy 100 to detect audio feedback from a user. The toy 100 may include a headphone jack. The headphone jack can facilitate transmission of audio sound to external sound devices such as speakers or headphones. The toy 100 may include a wireless connection 340. The wireless connection 340 is generally in the form of a Bluetooth connection. The wireless connection 340 enables a user and/or parent of a user to connect to the toy 100 via a mobile device. The wireless connection may also enable a user and/or parent of a user to connect to the speakers via a mobile device. As will be explained in more detail later, a user may connect the toy 100 to a mobile application (app) to adjust settings and program stimuli. In other examples, the toy 100 may include a heartrate monitor to send heartrate information to the app.

The toy 100 may optionally include one or more regions and/or zones 342. Generally, the toy 100 includes three regions 342 corresponding to three emotional states. The first region 345 generally corresponds to a fight or flight emotion, the second region 355 generally corresponds to a limbic system emotion, and the third region 365 generally corresponds to a calm/happy emotion. As has been mentioned previously, the regions may be color coded. For example, the first region 345 may be red, the second region 355 may be yellow, and the third region 365 may be green.

The toy 100 may optionally include one or more buttons 352. Generally, the toy 100 includes three buttons 352 corresponding to three emotional states. The first button 350 generally corresponds to a fight or flight emotion, the second button 360 generally corresponds to a limbic system emotion, and the third button 370 generally corresponds to a calm/happy emotion. As has been mentioned previously, the buttons may be color coded. For example, the first button 350 may be red, the second button 360 may be yellow, and the third button 370 may be green.

As should be appreciated, the buttons 352 may be associated with the zones 342. For example, the first region 345 may include the first button 350, the second region 355 may include the second button 360, and the third region 365 may include the third button 370. As has been mentioned previously, the buttons may be color coded to match the region. For example, the first button 350 may be red, the second button 360 may be yellow, and the third button 370 may be green. Generally, the buttons 352 work to activate an intervention from the corresponding regions emotional state. For example, pressing the third button 370 in the third region 365 would give a reward, such as a joke, story, favorite song, and/or other reward.

The toy 100 may optionally include a warming pad for generating heat to the toy.

The toy 100 may optionally include a removable cover for protecting the toy from dust. The removable cover may be washable.

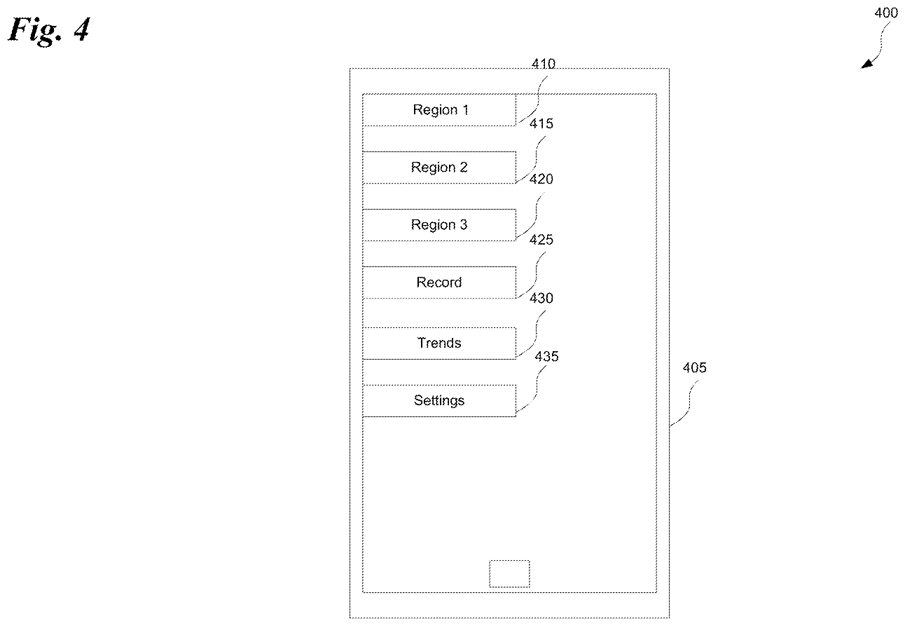

Illustrated in FIG. 4 is an example of a mobile application (app) 400 for use with the toy 100 described previously. The app 400 is generally located on a mobile device 405. The mobile device 405 may be a cell phone, tablet, computer, and/or any combination thereof. The app 400 may include a button 410 corresponding to the first region 345, a button 415 corresponding to the second region 355, and/or a button 420 corresponding to the third region 365. The buttons may be used to program and/or assign stimuli to each region. For example, clicking the button 415 for the first region 345 enables a user to assign intervention/stimuli to the first region 345. Optionally, the user may also remove stimuli from the first region 345 and/or set the region to alternate between certain stimuli randomly. As should be appreciated, the toy may also be used with a website and/or a remote control.

The app 400 may also include a button 425 for recording personal stimuli. For example, the user may record an audio clip of themselves reading a book, singing a song, saying positive affirmations, and/or any combination thereof. The recorded clip may then be assigned to one of the regions and used as an intervention.

The app 400 may include a trend button 430 shows statistical information related to the amount of times a child was in each zone, the amount of time it took a child to transfer into the next zone, the heartrate of the child in each zone, and other data related to behavioral statistics. As should be appreciated, the toy 100 may upload this data to the app 400 instantaneously and/or in a batch upload process once per day.

The app 400 may include a settings button 435 enables a user to adjust settings of the toy 100. For example, the user may change the vibration intensity of the toy, the light brightness and/or color, check toy battery life, adjust the volume of the toy, and/or adjust other settings.

The app 400 may include one or more subscription services (not shown for ease of illustration). For example, the app 400 may include a subscription to pre-recorded and/or live positive motivations. In one example, the positive motivations may include the name of the child to stimulate an emotional response. In another example, the subscription may be for live two-way conversation. In yet another example, the subscription may include a pre-recorded reading and/or speaking in Thinker's, Jack's, and/or any other book characters voice. In a further example, the subscription may include additional interventions, such as daily assignments. An example of a daily assignment may be to find ______ today, walk ______ steps, perform ______ exercises, and/or other assignments/interventions.

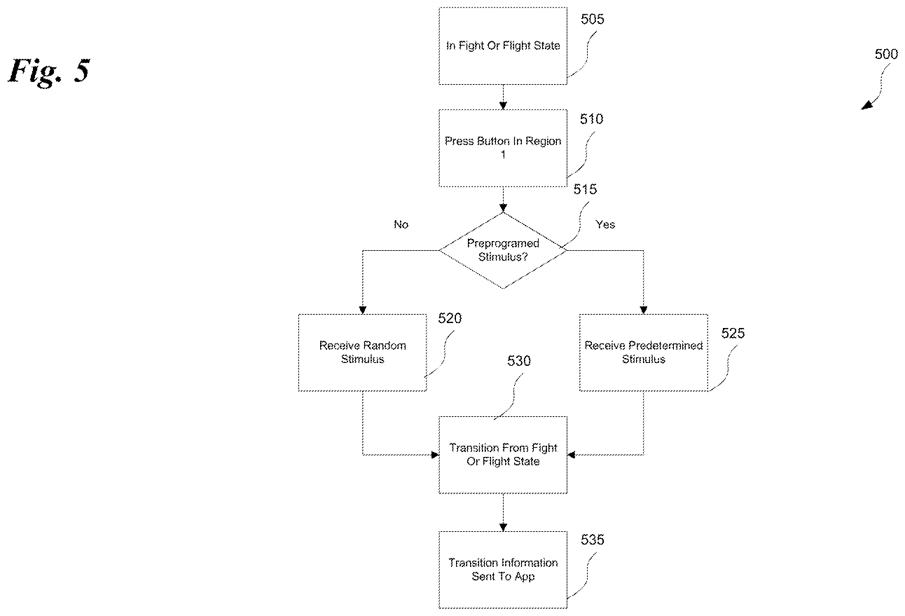

An example technique for using the toy 100 will now be described with respect to FIGS. 1-4. Shown in FIG. 5 is a flowchart 500 of a portion of the self-calming process. At stage 505 the child is in a fight or flight emotional state. For example, the child may be angry, upset, sad, and/or a feeling a similar emotion. At stage 510, the child presses the button 350 in the first and/or red region 345. In other examples, a parent and/or caregiver may press the button via a remote control. Generally, the button 350 will be either illuminated red via one or more lights and/or a red colored material. At stage 515 the controller 305 of the toy 100 determines whether the toy is set-up to deliver a preprogrammed intervention via settings on the app 400. If not, the child receives a randomly generated intervention at stage 520. The random intervention may be vibration, calming music, meditative music, nature sounds, instructions to narrow focus, deep breathing exercise, crossing the midline exercises, instructions to perform manual exercise, hugging/petting the toy, progressive muscle relaxation, meditation, counting exercises, and/or other calming strategies. If the toy 100 is programmed to deliver a preprogrammed intervention, the preprogrammed intervention is delivered at stage 525.

At stage 530 the child begins to calm and transfer from the fight or flight emotional response down into the yellow or limbic system response. In some examples, the child may transfer from the red or fight or flight response directly to the green or calm emotional state. In other examples, the child may remain in the fight or flight state and restart the calming process at stage 510.

At stage 535, the controller 305 sends the emotional transition information to the app. The controller 305 may send information related to the number of times the child was in each zone and/or the amount of time for the child to transfer out of the zone.

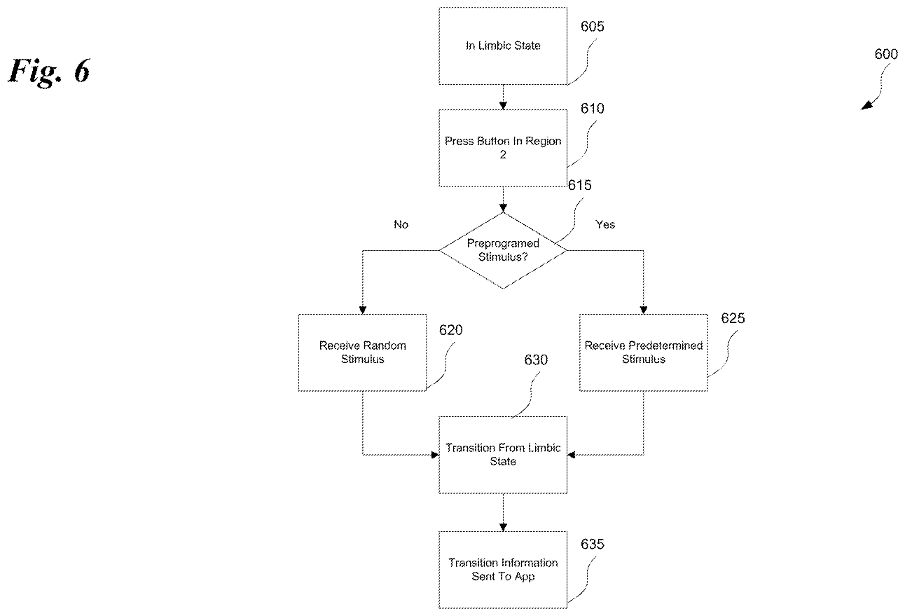

Shown in FIG. 6 is a flowchart 600 depicting a technique for transitioning from the limbic state into the calm/happy state. At stage 605 the child is in the yellow or limbic emotional state. For example, the child may have started to calm down from the red or fight or flight state. In another example, the child may be in the green or calm state and begin to get upset. When a child feels this transition in mood the child and/or the user presses the button 360 in the second or yellow region as shown in stage 610. Generally, the button 360 will be either illuminated yellow via one or more lights and/or a yellow colored material. At stage 615 the controller 305 of the toy 10 determines whether the toy 100 is set-up to deliver a preprogrammed intervention via settings on the app 400. If not, the child receives a randomly generated intervention at stage 620. The random intervention may be telling a story, imagining a safe place, EFT tapping, positive affirmations, deep breathing exercises, motivational statements, journaling prompts, jokes, and/or other calming strategies. If the toy 100 is programmed to deliver a preprogrammed intervention, the preprogrammed intervention is delivered at stage 625.

At stage 630 the child begins to calm and transfer from the limbic emotional response down into the green or calm/happy system response. In some examples, the child may remain in the limbic state and restart the calming process at stage 610. In other examples, the child may be unable to calm themselves and escalate into the red or fight or flight response.

At stage 635, the controller 305 sends the emotional transition information to the app 400. The controller 305 may send information related to the number of times the child was in each zone, the heartrate of the child in each zone, and/or the amount of time for the child to transfer out of the zone.

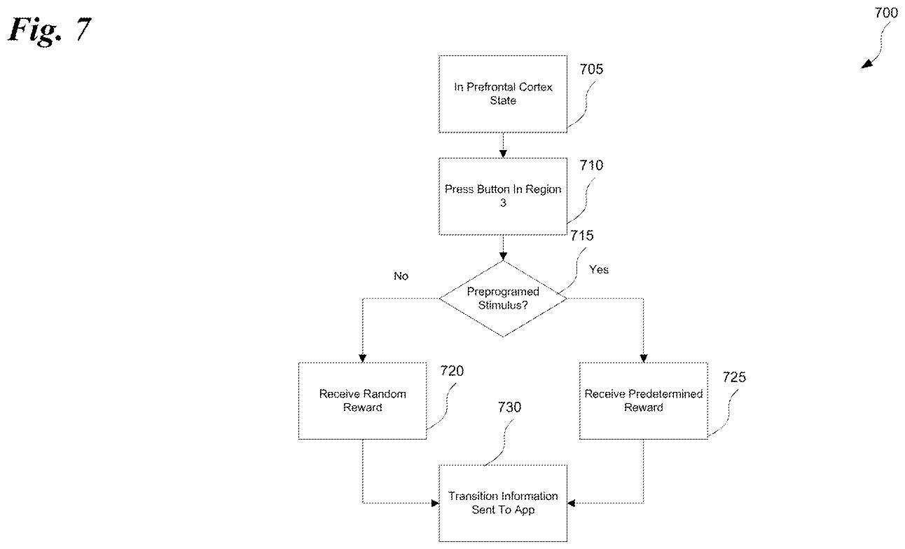

Shown in FIG. 7 is a flowchart 700 depicting a technique for rewarding a child for reaching the green or calm/happy state. At stage 705 the child is in the green or calm emotional state. For example, the child may have started to calm down from the limbic state. When a child feels this transition in mood the child presses the button 370 in the third or green region 365 as shown in stage 710. In other examples, a parent and/or caregiver may press the button via a remote control. Generally, the button 370 will be either illuminated green via one or more lights and/or a green colored material. At stage 715 the controller 305 of the toy 100 determines whether the toy 100 is set-up to deliver a preprogrammed reward via settings on the app 400. If not, the child receives a randomly generated reward at stage 720. The random reward may be a light show with congratulations, a favorite song, a new song, a story, jokes, and/or other rewards. If the toy 100 is programmed to deliver a preprogrammed reward, the preprogrammed reward is delivered at stage 725.

At stage 730, the controller 305 sends the emotional transition information to the app 400. The controller 305 may send information related to the number of times the child was in each zone, the heartrate of the child in each zone, and/or the amount of time for the child to transfer out of the zone.

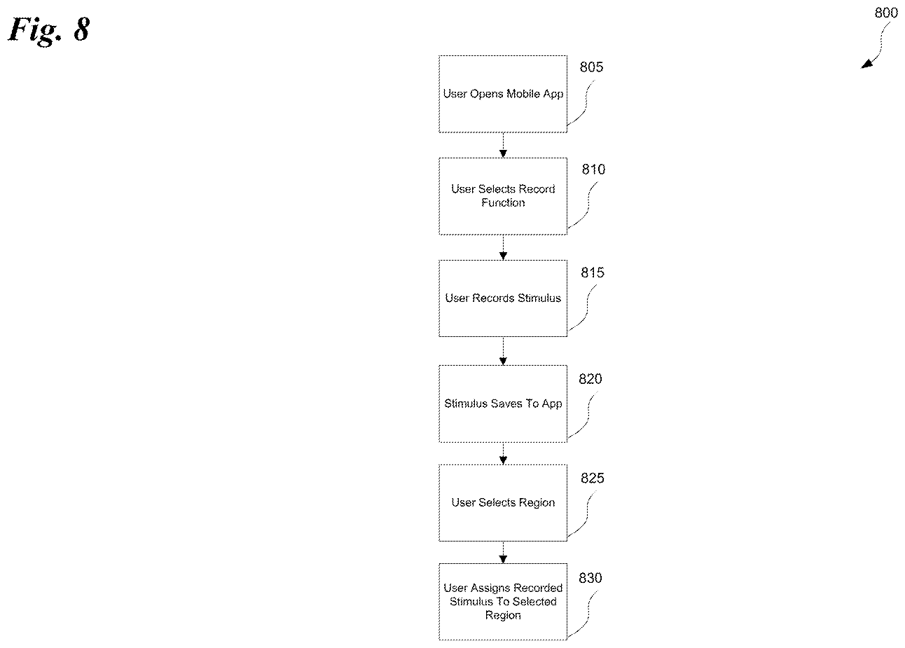

Shown in FIG. 8 is a flowchart 800 depicting a technique for recording an intervention for use with the toy 100 described previously. At stage 805 the user opens the app 400 associated with the toy 100 as described best in FIG. 4. At stage 810 the user selects the record function 425 within the app 400. At stage 815 the user records a personal intervention and/or reward. For example, the user may record a song, reading a book, praise, and/or any other intervention and/or reward. At stage 820 the app 400 saves a recording of the intervention/reward. At stage 825 the user selects a region 342 to assign the intervention/reward to. For example, the user may assign the intervention to the first and/or red region 345 to help in self-calming. At stage 830, the user confirms the assignment of the intervention/reward to the region.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, “left”, “right”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

The following numbered clauses set out specific embodiments that may be useful in understanding the present invention:

1. A method of using an interactive emotional support device, comprising:
pressing a first button in a first region of the interactive emotional support device having a controller, a speaker, a motor, and first and second buttons in different zones when experiencing an automatic physiological reaction;
receiving one or more interventions from a first group of interventions of the interactive emotional support device;
repeating the one or more interventions from the first group of interventions;
pressing the second button after beginning to calm down from the automatic physiological reaction; and
receiving one or more interventions from a second group of interventions of the interactive emotional support device;
wherein the first group of interventions include at least one selected from the group consisting of: guided breathing exercises, guided physical exercises, guided activities with the interactive emotional support device, guided muscle relaxation, guided counting exercises, guided storytelling, guided visualization, guided acupressure, guided positive statements, and guided journaling; and
wherein the second group of interventions include at least one selected from the group consisting of: telling a joke, providing a light display, playing a song, and telling a story.

2. The method of clause 1, further comprising:
pressing a third button of the device after pressing the first button and before pressing the second button; and
receiving one or more interventions from the first group of interventions of the interactive emotional support device and different from the other receiving one or more interventions from a first group of interventions of the interactive emotional support device.

3. The method of any preceding clause, wherein the device is a stuffed brain-shaped toy.

4. The method of any preceding clause, wherein the device is weighted to at least 5 pounds.

5. The method of any preceding clause, wherein the first button is colored red, wherein the second button is colored yellow, and wherein the third button is colored green.

6. The method of any preceding clause, wherein the interactive emotional support device communicates with a mobile application (app) via a wireless connection.

7. The method of clause 6, wherein the app enables a user to assign specific interventions to the first and/or second groups of interventions.

8. The method of clause 6, wherein the app tracks the number of times that a user presses the first, second and/or third buttons.

9 The method of clause 6, wherein the app tracks the amount of time between presses of the first and second buttons, between presses of the first and third buttons, and/or between presses of the third and second buttons.

10. The method of clause 6, wherein the app enables a user to record interventions, and wherein the user can then assign the intervention to the first and/or second group of interventions via the app.

11. A method of using a stuffed brain-shaped interactive emotional support device having a controller, one or more speakers, a motor, a microphone, one or more lights, a wireless connection, and one or more buttons corresponding to one or more zones, comprising:
pressing a first button in a first region of the device corresponding to a red and/or fight or flight emotional state when in the fight or flight emotional state; and,
receiving and following a preprogrammed intervention from the device based on the fight or flight emotional state;
wherein the device is a stuffed brain-shaped toy, wherein the device communicates with a mobile application (app) via a wireless connection, and wherein the preprogrammed interventions are assigned to one or more buttons via the app.

12. The method of clause 11, further comprising:
pressing a second button in a second region of the device corresponding to a yellow and/or limbic emotional state when in the limbic state; and
receiving and following an intervention from the device base on the limbic state.

13. The method of any one of clauses 11-12, further comprising:
pressing a third button in a third region of the device corresponding to a green and/or calm emotional state when in the calm state; and,
receiving a reward from the device based on the calm state.

14. The method of any one of clauses 11-13, wherein the device is weighted to at least 3 pounds.

15. The method of clause 13 or 14, wherein the first button is colored red, wherein the second button is colored yellow, and wherein the third button is colored green.

16. The method of any one of clauses 13-15, wherein the app tracks the number of times that a user enters the red, yellow, and/or green zones.

17. The method of any one of clauses 12-16, wherein the app tracks the amount of time for a user to transfer between the red and yellow zones.

18. The method of any one of clauses 13-17, wherein the app tracks the amount of time for a user to transfer between the yellow and green zones.

19. The method of any one of clauses 11-18, wherein the app enables a user to record personalized intervention, and wherein the user can then assign the personalized intervention to the preprogrammed intervention via the app.

20. The method of any preceding clause, wherein the device is moveable from a play arrangement to a travel pillow arrangement via one or more fasteners.

### Glossary of Terms

The language used in the claims and the written description is to only have its plain and ordinary meaning, except for terms explicitly defined below. Such plain and ordinary meaning is defined here as inclusive of all consistent dictionary definitions from the most recently published (on the filing date of this document) general purpose Merriam-Webster dictionary.

As used in the claims and the specification, the following terms have the following defined meanings:

“And/or” is inclusive here, meaning “and” as well as “or”. For example, “P and/or Q” encompasses, P, Q, and P with Q; and, such “P and/or Q” may include other elements as well.

“Button” generally refers to an electrical switch. A button is typically used to complete, change, and/or break an electrical circuit. Buttons may be a variety of shapes from rectangular, triangular, and/or circular to name a few. In other embodiments, a button may have a fanciful shape, such as an animal shape or the shape of an object. In further embodiments, the button may be colored.

“Color” means the predominant light color (for example, red, amber, white, blue, green, yellow, purple, or otherwise) even if it is blended with some non-predominant light wave or frequency light.

“Controller” generally refers to a mechanical or electronic device configured to control the behavior of another mechanical or electronic device. A controller may include a “control circuit” configured to provide signals or other electrical impulses that may be received and interpreted by the controlled device to indicate how it should behave.

“Fastener” generally refers to a hardware device that mechanically joins or otherwise affixes two or more objects together. By way of non-limiting examples, the fastener can include bolts, dowels, nails, nuts, pegs, pins, rivets, screws, hook and loop fasteners, and snap fasteners, to just name a few.

“Fight or Flight” generally refers to a response that is an automatic physiological reaction to an event that is perceived as stressful or frightening.

“Hook and Loop Fastener” generally refers to two separated components, the hooks and the loops. When the hooks and loops are pressed together, the hooks capture the loops and create a removable connection. To separate the hooks and the loops, a pulling or peeling force is required. Hook and loop fasteners may come in a variety of shapes and sizes. For example, the hook and loop fasteners may be rectangular, circular, and/or a variety of other shapes depending on the use case. Generally, hook and loop fasteners are secured to opposing surfaces via an adhesive and/or sewing.

“LED” means light emitting diode, including single diodes as well as arrays of LED's and/or grouped light emitting diodes. This can include the die and/or the LED film or other laminate, LED packages, said packages may include encapsulating material around a die, and the material, typically transparent, may or may not have color tinting and/or may or may not have a colored sub-cover. An LED can be a variety of colors, shapes, sizes and designs, including with or without heat sinking, lenses, or reflectors, built into the package.

“Light Color” means the predominant light color visible to the naked human eye (for example, red, amber, white, blue, green, yellow, purple, or otherwise) even if it is blended with some non-predominant light wave or frequency light.

“Limbic Response” generally refers to an emotional response from within the limbic system, specifically the amygdala. However, the limbic response is generally less severe and emotional than the fight or flight response.

1.-20. (canceled)

21. An interactive device operable to teach a user self-regulation comprising:
a body configurable to house a controller for operating the interactive device, and at least one of a speaker and a headphone jack; and
three interactive components associated with the body wherein a first interactive component is operable to initiate delivery of a first intervention configured to stimulate a vagus nerve of the user upon activation, a second interactive component is operable to initiate delivery of a second intervention configured to promote a switch from a sympathetic nervous system to a parasympathetic nervous system of the user upon activation, and a third interactive component is operable to initiate delivery of a third intervention configured to stimulate the parasympathetic nervous system upon activation, further wherein the first intervention is delivery of a calming intervention, the second intervention is delivery of a processing intervention, and the third interventions is delivery of a reward intervention.

22. The interactive device of claim 21 further comprising one or more of vibrating motors, lights, and microphones.

23. The interactive device of claim 21 wherein each of the three interactive components is operable to initiate at least one of a visible intervention, an audible intervention, and a tactile intervention.

24. The interactive device of claim 21 wherein each of the three interactive components is a at least one of a button, a shape, and a texture.

25. The interactive device of claim 21 further comprising an attachment operable to deliver aroma.

26. The interactive device of claim 21 further comprising one or more of a warming pad, a weight, and a removable cover.

27. The interactive device of claim 21 further comprising a timer configurable to measure a time between user inputs.

28. The interactive device of claim 21 further comprising an app in wireless communication with the device.

29. The interactive device of claim 28 wherein the app in wireless communication with the device is operable to remotely control the interactive device.

30. The interactive device of claim 21 further comprising a recorder operable to record one or more of emotion data and physical data.

31. A method of teaching a user self-regulation comprising:
providing an interactive device operable to teach a user self-regulation having a body configurable to house a controller for operating the interactive device, and at least one of a speaker and a headphone jack, and three interactive components associated with the body wherein a first interactive component is operable to initiate delivery of a first intervention configured to stimulate a vagus nerve of the user upon activation, a second interactive component is operable to initiate delivery of a second intervention configured to promote a switch from a sympathetic nervous system to a parasympathetic nervous system of the user upon activation, and a third interactive component is operable to initiate delivery of a third intervention configured to stimulate the parasympathetic nervous system upon activation, further wherein the first intervention is delivery of a calming intervention, the second intervention is delivery of a processing intervention, and the third interventions is delivery of a reward intervention;
selecting at least one of the first interactive component, the second interactive component, and the third interactive component; and
delivering at least one of the first intervention in response to selection of the first interactive component, the second intervention in response to selection of the second interactive component, and the third intervention in response to selection of the third interactive component

32. The method of claim 31 wherein the first intervention is one or more of guided breathing exercises, guided physical exercises, guided activities with the interactive emotional support device, guided muscle relaxation, guided counting exercises, guided storytelling, guided visualization, guided acupressure, guided positive statements, and guided journaling

33. The method of claim 31 wherein the second intervention is one or more of playing a joke, displaying lights, playing a song, and playing a story.

34. The method of claim 31 further comprising receiving instructions wirelessly.

35. The method of claim 31 further comprising recording emotional data or physical data.

36. The method of claim 31 further comprising assigning preprogrammed stimuli to one or more of the first interactive component, the second interactive component, and the third interactive component.
